(19) United States
(12) Patent Application Publication
(10) Pub. No.: US 2023/0041330 A1
(43) Pub. Date: Feb. 9, 2023

(54) INTRAOCULAR SECONDARY LENS AND APPLICATION METHODS THEREOF THAT CAN BE ADHERED ON THE INTRAOCULAR LENS IN PSEUDOPHAKIC EYES

(71) Applicant: ONDOKUTZ MAYIS UNIVERSITESI REKTORLUK, Samsun (TR)

(72) Inventor: Volkan YETER, Samsun (TR)

(21) Appl. No.: 17/789,865

(22) PCT Filed: Dec. 29, 2020
(86) PCT No.: PCT/TR2020/051433

§ 371 Date: Jun. 29, 2022

(30) Foreign Application Priority Data
Dec. 30, 2019 (TR) 2019/22569

(51) Int. Cl. A61F 2/16; A61F 2/00

(52) U.S. Cl. CPC A61F 2/1602; A61F 2/0077; A61F 2/1651

(57) ABSTRACT

The present invention relates to an intraocular secondary lens (L) for insertion into the eye other than the lens that is implanted in the eye during cataract surgery so as to change the refractive power and/or to change the direction and shape of the image rays entering the eye in the patient who have undergone cataract surgery and to whom intraocular lenses are inserted. The secondary lens (L) is in a form that can be easily adhered on the primary lens (M) or the capsule (4) in which the primary lens is located and be easily removed from thereto, it has a foldable feature and contains adhesive nanostructures (6) thereon. It can be easily applied to the eye without need for structures such as hole, notch, foot etc. on the primary lens (M) with the invention by means of the nano structures (6) on the secondary lens. Said secondary lens (L) may be in the form of normal refractive, diffractive, accommodative, and toric, trifocal, multifocal, or combinations thereof, or optionally may carry devices with different optical properties.

### FIELD OF THE INVENTION

The present invention relates to an intraocular secondary lens for insertion into the eye other than the lens that is implanted in the eye during cataract surgery so as to change the refractive power and/or to change the direction and shape of the image rays entering the eye in the patient who have undergone cataract surgery and to whom intraocular lenses are inserted.

### STATE OF THE ART

There is a transparent lens/lens in the eye that provides the eye to focus on objects and can be able to change its refraction while focusing on the object to be seen. The lens which loses its transparency depending on many reasons such as some diseases and drugs, radiation, trauma and particularly aging etc. opacifies such that it prevents the passage of the image to the perceptual retina layer. This opacification in the lens is called “cataract”. The treatment of cataract is performed by a surgical operation; the lens of the patient that has been in the eye as of birth is removed and an artificial lens (intraocular lens) is implanted instead of this lens.

The primary lens, which is implanted in the eye during cataract surgery, generally consists of legs (haptic), the upper and lower, which basically keep the lens perpendicular to the optical plane, and an optical plane with optical refraction. The optical plane provides clear view by dropping the image onto the light-perceiving macula. Generally, the primary lenses implanted in the eye are monofocal, and thus here it is usually aimed to drop the rays coming from infinity to the macula so as to keep the distance viewing at a good level. A small amount of the primary lenses implanted in cataract surgery are the lenses that are known to correct astigmatism (toric) and to correct both near and far (multifocal). The primary lens implanted in the eye by a surgery without complication is implanted in a thin transparent membrane called the capsule that surrounds the lens on all sides and it replaces the patient's organic lens, which was removed with cataract. After this primary intraocular lens implantation, the patient may require high or undesired glasses (refraction defect) depending on miscalculations or various reasons. One of the methods used to correct this is carried out by placing a secondary lens in front of the primary lens. This process is called piggyback intraocular lens implantation or secondary intraocular implantation.

The capsule is opened in a circular manner from the front section of the capsule during cataract surgery and the cataract is removed from this gap with the help of special tools and methods. Only a structure having a bag shape with a thin transparent membrane wall opened in circular form is remained. The visual level is attempted to be brought closer to normal by implanting intraocular artificial lenses from different materials with different refraction and structure in this cavity that will substitute the position and function of the organic lens of the patient.

Measurements are made prior to the surgery and it is calculated how many diopters of power that the lens implanted in the patient's eye will involve. However, there may be mistakes in a ratio of 15-20% in the calculations and the patients need glasses at an undesirable level after surgery. Various surgical methods are applied in the state of the art so as to solve this problem permanently. Widespread methods applied in the state of the art are as follows:
a) The lens which is implanted in the eye during surgery is removed in an early period (before the lens adheres to the capsule so much), a lens with proper refraction is implanted instead of this. Tears may occur in the capsule where the lens is implanted during this intervention and thus this may lead to many complications.
b) The remaining refractive defect can be corrected by means of corneal laser applications, but the corneal structure of every patient may not be appropriate for this method.
c) A secondary intraocular lens is implanted in front of the lens implanted in the eye during cataract surgery, namely in the area called the chamber between the iris and the capsule wherein the intraocular artificial lens is implanted. The legs of the lens implanted in this area are in contact with an area called “ciliary sulcus” that is close to the rear root of the iris. Therefore two overlapping lenses fulfil the necessary refractive requirement. Other than correcting the refractive surprises caused by incorrect measurements; also the multifocal lens, which allows the patient to see both near and far, and toric lenses to correct high astigmatism can be realized with the help of the secondary intraocular lens. This implantation method is also known as secondary (secondary) intraocular lens implantation, piggyback lens implantation or dual lens implantation. The legs of the implanted secondary lens, called haptic, increase the eye pressure by contacting the rear section of the iris root and sometimes iridocorneal angle or may lead to problems such as iris atrophy, pigmentary glaucoma, opacification between both lenses, and forward slip of the secondary lens. Products such as Add-on, 1st Add-on, and Sulcoflex have been produced in the state of the art by improving the angle of the leg structure and optical center of the secondary piggyback lens so as to reduce such problems.

In the state of the art, the secondary lens is attached to the primary lens by means of some needles, pins, clips, and/or fasteners. Furthermore, prior to this process, holes are made in the primary lens in the eye or a primary lens with specific holes is implanted in the first surgery. Subsequently, the secondary lens is implanted in front of the primary lens by means of some pins, clips or fasteners.

Due to the difficulty and insufficiency of the current solutions, a development is required to be made in the relevant technical field. Said invention relates to a secondary lens developed for use in patients who have undergone cataract surgery and to whom intraocular lenses are implanted and an application method thereof so as to eliminate the disadvantages of the state of the art.

### BRIEF DESCRIPTION OF THE INVENTION AND AIMS OF THE INVENTION

In the present invention, an adhesive/removable secondary lens so as to be applied on the primary lens in the eye or on the capsule where the primary lens is found, in patients who have undergone cataract surgery and have an intraocular lens (primary lens) implanted in their eye is disclosed

An aim of the invention is to develop a secondary lens for applying the same on the primary lens in the eye without making any change on the primary lens or on the capsule in which the primary lens is contained.

Another aim of the invention is to eliminate the requirement of high or undesired glasses (refraction defect) in the patient after this primary intraocular lens implantation, depending on miscalculations or various reasons.

In the present invention, no change is made in the form and/or structure of the primary lens that is implanted into the eye in the first surgery; no holes or notches are made on the primary lens.

Another aim of the invention is to implant the same in the eye on the primary lens or on the capsule containing the primary lens without requiring different auxiliary clips or lens legs. The adhesion of the secondary lens on the surface where it is applied is performed by means of the nano/micro structures on the secondary lens, or on a part holding secondary lens (for example: a frame). While the secondary lens (L) is placed on the primary lens (M), any further connection element is not required, or a holder/frame adhered over the primary lens. This holder/frame can hold the secondary lens in a proper position over the primary lens by the adhesion force of nano/micro structures.

Another aim of the invention is to provide an adhesive and removable secondary lens that can be easily attached/adhered to and removed from the primary lens or the capsule in which the primary lens is contained. In the present invention, the secondary lens can be adhered on, removed from and re-adhered on the primary lens or on the capsule in which the primary lens is contained like a sticker. Said secondary lens can be easily replaced with different lenses since it can be easily adhered and removed. Particularly, in the lens implantation with toric namely astigmatism, rotation of the lens and placing the same in the suitable axis is important.

By means of the invention, the optical axis of the secondary lens (L) can be adjusted easily and its location can be easily changed because it can be attached and detached in patients having the primary lens with a high kappa angle and where multifocal lenses cannot be implanted.

There are adhesive nanostructures that ensure the primary lens to adhere onto the smooth surface of the primary lens or onto the surface of the capsule containing the primary lens in the present invention and different adhesion ability can be achieved by changing the structure, shape and number of these nano/micro structures. The secondary lens can be designed as two or more parts containing nano/micro structures and can be implanted over the primary lens in multiple steps. By these adhesive parts may contain different refractive or cosmetic parts.

### DESCRIPTION OF THE FIGURES

In order to understand the advantages of the present invention with its structure and additional elements, it shall be evaluated with the following defined figures.

FIG. 1: Front view of the primary intraocular lens (A), (B) front view (C) after insertion into the capsular structure, (C) transverse view of its status in the eye after insertion into the capsule.

FIG. 2: 360° location of adhesive nanostructures in the adhered secondary lens.

FIG. 3: view of secondary lenses in different structures when adhered on primary lens (A, B) and capsule (C).

FIG. 4: view of the locations of adhesive nanostructures dispersed over the secondary lens.

FIG. 5: view of the adhesive nanostructures on the secondary lens when they are dispersed as segmented and adhered onto the primary lens.

FIG. 6: view of the primary lens when it adheres on the capsule where it is found, by means of adhesive nanostructures placed on the feet on the secondary lens.

FIG. 7: side view of the secondary lens when adhered on the primary lens.

FIG. 8: view of adhesive nanostructures mounted on or produced on the secondary lens.

FIG. 9: views of (A) Spherical, (B) Toric and (C) Multifocal secondary lenses.

FIG. 10: Views of the secondary lens (L) implanted by the frame attached on the primary lens (M) or the capsule (4).

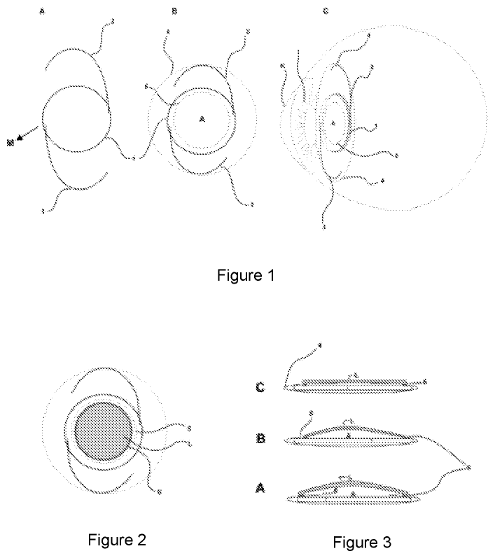

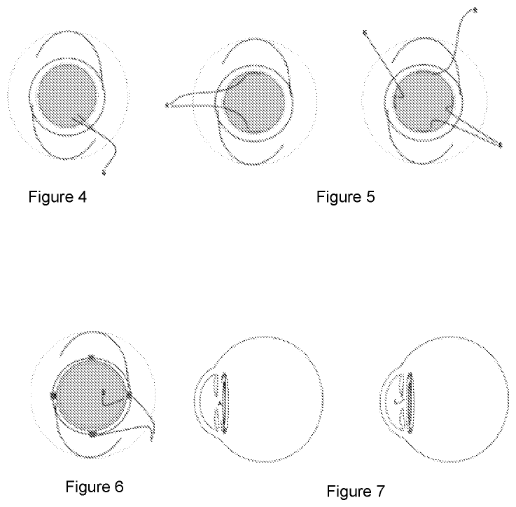

### DESCRIPTION OF THE REFERENCES

The features of the invention in the figures are enumerated and the corresponding numbers are given below in order to explain the secondary lens developed with this invention better:
1. Optical plane with refraction of primary lens
2. Upper leg
3. Lower leg
4. Capsule/bag in which the primary lens is implanted
5. Front capsule border
6. Nano/micro structure
7. Foot with nanostructure
8. Frame
A. Front capsule opening
I. Iris
K. Cornea
L. Secondary lens
M. Primary lens

### DETAILED DESCRIPTION OF THE INVENTION

The present invention relates to an adhesive/removable secondary lens so as to be applied on the primary lens or the capsule (4) containing the primary lens, in patients who have undergone cataract surgery and have an intraocular lens (primary lens) implanted in their eye. This intraocular secondary lens comprises nano/micro structures (6) in adhesive form on it so as to attach to the surface where it is implanted. Said secondary lens (L) comprises nano/micro structures (6) on the lower contact surface and/or the upper contact surface so as to adhere on the surface where it is implanted.

The secondary lens (L) may comprise nano/micro structures (6) on the lower surface only contacting the primary lens (M) or may comprise nano/micro structures (6) both on the lower surface contacting the primary lens (M) and on the upper surface contacting the capsule (4) in case that the secondary lens (L) is implanted on the primary lens (M) in the capsule (4). The secondary lens (L) comprises nano/micro structures (6) on its lower surface only in contact with the capsule (4) in case it is located on the capsule (4).

The intraocular secondary lens (L) subjected to the invention is in foldable form and is made of different flexible materials such as acrylic, silicone or hydrogel. There are nano/micro structures (6) providing adhesion on the surface where the secondary lens (L) contacts with the primary lens (M) or on the surface where it contacts with the capsule (4) containing the primary lens. As in FIGS. 2 and 3, these can be placed on the surface by different manners or methods and they are placed on the legs extending from the secondary lens and adhered onto the capsule (FIG. 3-C).

The diameter of the intraocular secondary lens (L) is between 3-7 mm. Therefore, it can be adhered on the lens optics (FIG. 7) only through the front capsule opening (A), also it can be adhered on capsule in the periphery.

The optics of the intraocular secondary lens (L) can be hydrophobic or hydrophilic.

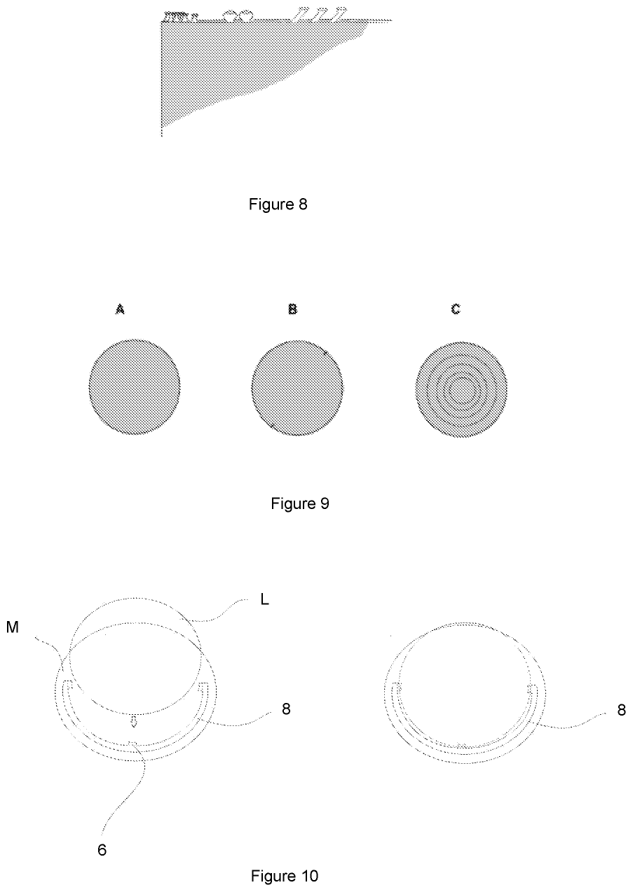

The nanostructures in the secondary lens (L) can be originated from nature such as octopus suction cups or Tokay gecko foot holder etc. or can have different technical shapes (FIG. 8). The secondary lens (L) can adhere onto the primary lens or to the capsule tissue in which it is found by using any physical adhesion that has an adhesive effect such as hydrophobic-hydrophobic bonds, Van der Waals bonds, electrostatic bonds, vacuum force etc. and they can be adhered and removed like reusable double-sided stickers or Nano gel-like strip tapes.

Nano/micro structures (6) have a dimension between 1 nm-100 mm by specific methods such as electron microscope lithography and can be produced with desired shape and structure on the lens and can be implanted on the secondary lens (L) after its production. More nano/microstructures (6) can be implanted on the surface of the secondary lens (L) since the nano/micro structures (6) have such dimensions, therefore the adhesion strength on the surface to which the secondary lens (L) will be adhered increases. The location and configuration of the nanostructures (6) on the secondary lens (L) can be changed as required. Also, these nano/micro structures (6) can be produced with different methods, and they can affect the passage of the rays forming the vision in a small and imperceptible manner since they are very small structures, this difference can be ignored.

Nano/micro structures that can be produced from processable biocompatible polymers such as hydrophobic, hydrophilic or superhydrophobic siloxane, polyurethane, acrylic etc. nano/micro structures (6), can be made by nanotechnological methods such as two-photon lithography, electron microscope lithography, electrospin method, ultraviolet inking-printing-curing, plasma abrasion, colloidal assembly and casting, other casting methods etc. and different forms can be given to these nano/micro structures (6).

Said secondary lens (L) may be in the form of normal refractive, diffractive, accommodative, toric, trifocal, multifocal, or combinations thereof, or optionally may comprise devices with different optical properties or may be produced for different cosmetic purposes (for example: iris prosthesis) (FIG. 9). Furthermore, the secondary lens (L) can be injected into the eye through small incisions by means of different injectors or can be easily removed from the eye since it is foldable.

The optical axis of the secondary lens (L) can be adjusted easily and its location can be easily changed because it can be attached and detached. While the secondary lens (L) to be placed on the primary lens is held on the primary lens (M) by means of the nano/micro structures (6), this implantation procedure can be performed with more than one content and with more than one step. In an embodiment of the invention; the secondary lens (L) can be implanted and removed by means of a frame (8) bonded with nano/micro structures on the primary lens (M) or on the capsule (4) containing the primary lens. This frame (8) can be produced in different shapes and structures in accordance with different secondary lens (L) structures. The secondary lenses (L) with different properties can be easily implanted and removed from the frame (8) placed on the primary lens (M) or the capsule (4). The frame (8) can stay on the eye continuously, and the optical mechanism to be placed on it can be changed many times. Thus, possible trauma on the lens is minimized and multiple lens changes can be easily achieved with a single adhesion (FIG. 10).

1. An intraocular secondary lens (L) in the form that is directly implantable on the primary lens or on the capsule (4) containing the primary lens, without any deformation and without making any hole or notching on the primary lens, for use in pseudophakic eyes that have undergone cataract surgery and have had an intraocular lens (primary lens) implanted in the eye, characterized in that; it comprises adhesive nano/micro structures (6) in removable form on the lower contact surface and/or upper contact surface to adhere on the surface where it is implanted.

2. An intraocular secondary lens (L) according to claim 1, characterized in that; it comprises nano/micro structures (6) only on the lower surface contacting the primary lens (M) or on the lower surface contacting the primary lens (M) and on the upper surface contacting the capsule (4) in case the secondary lens (L) is applied on the primary lens (M) in the capsule (4).

3. An intraocular secondary lens (L) according to claim 1, characterized in that; it comprises nano/micro structures (6) on its lower surface only in contact with the capsule (4) in case secondary lens (L) is located on the capsule (4).

4. An intraocular secondary lens (L) according to claim 1, characterized in that; the secondary lens (L) comprises nano/structures (6) with which adhesion to the surface it is placed is provided by using any physical adhesion that has an adhesive effect such as hydrophobic-hydrophobic bonds, Van der Waals bonds, electrostatic bonds, vacuum force etc. and that can be placed on the surface in various forms or methods.

5. An intraocular secondary lens (L) according to claim 1, characterized in that; the secondary lens (L) is foldable.

6. An intraocular secondary lens (L) according to claim 1, characterized in that; the secondary lens (L) is made of flexible materials.

7. An intraocular secondary lens (L) according to claim 1, characterized in that; the secondary lens (L) is made of acrylic, silicone or hydrogel.

8. An intraocular secondary lens (L) according to claim 1, characterized in that; the optics of the secondary lens (L) is hydrophobic or hydrophilic.

9. An intraocular secondary lens (L) according to claim 1, characterized in that; the diameter of the secondary lens (L) is between 3-7 mm.

10. An intraocular secondary lens (L) according to claim 1, characterized in that; the secondary lens (L) is in the form of normal refractive, diffractive, accommodative, toric, trifocal, multifocal, and/or combinations thereof.

11. An intraocular secondary lens (L) according to claim 1, characterized in that; the secondary lens (L) comprises apparatus with different optical properties.

12. An intraocular secondary lens (L) according to claim 1, characterized in that; nano/micro structures (6) are between 1-100 microns.

13. An intraocular secondary lens (L) according to claim 1, characterized in that; nano/micro structures (6) are made of hydrophobic, hydrophilic or superhydrophobic processable biocompatible polymers.

14. An intraocular secondary lens (L) according to claim 1, characterized in that; it comprises nano/micro structures (6) that can be made by any of the following methods; two-photon lithography, electron microscope lithography, electrospin method, ultraviolet inking-printing-curing, plasma abrasion, colloidal assembly and casting.

15. An intraocular secondary lens (L) according to claim 1, characterized in that; it comprises a frame (8) bonded with nano/micro structures on the primary lens (M) or on the capsule (4) containing the primary lens wherein the secondary lens is implanted on the primary lens (M) or on the capsule (4) by means of the frame (8).
